(19) United States
(12) Patent Application Publication
(10) Pub. No.: US 2011/0166568 A1
(43) Pub. Date: Jul. 7, 2011

(54) NEUTRAL ELECTRODE DEVICE, ELECTROSURGICAL INSTRUMENT COMPRISING A CORRESPONDING NEUTRAL ELECTRODE DEVICE, CONTACT MEDIUM, AND USE OF A LATENT HEAT ACCUMULATOR FOR COOLING AN ELECTRODE

(76) Inventors: Martin Hagg, Wannweil (DE); Peter Selig, Hechingen (DE)

(21) Appl. No.: 13/062,755

(22) PCT Filed: Aug. 26, 2009
(86) PCT No.: PCT/EP09/06186

§ 371 Date: Mar. 8, 2011

(30) Foreign Application Priority Data
Sep. 9, 2008 (DE) 10 2008 046 300.0

(51) Int. Cl. A61B 18/16

(52) U.S. Cl. 606/37; 606/32

(57) ABSTRACT

In surgery using monopolar HF (high frequency), there is a constant risk of patients suffering burns at the neutral electrode. The problem stems from the fact that numerous methods have been devised by which relatively high HF currents are applied for extended periods of time. The present disclosure solves said problem by providing a neutral electrode device to be used for applying an HF current to a biological tissue. The improved neutral electrode device comprises at least one latent heat accumulator for absorbing heat. Thus, heat peaks can be at least temporarily accumulated until the accumulated thermal energy can be safely released.

### FIELD OF THE INVENTION

Embodiments of the invention relate to a neutral electrode device comprising a latent heat accumulator, an electrosurgical instrument with a corresponding electrode device, a contact medium with a latent heat accumulator and the use of a latent heat accumulator for cooling an electrode.

### BACKGROUND

In high frequency surgery (HF surgery), alternating currents are passed through the human body at high frequency to damage or cut tissues in a targeted way. A substantial advantage over conventional scalpel cutting techniques is provided because suppression of bleeding can take place simultaneously with the incision by closing the relevant vessels.

A monopolar technique is often used. With such a technique, one pole of the HF voltage source is connected to the patient over the largest area possible. This electrode is known as the neutral electrode. The other pole (the active electrode) is situated on a surgical instrument. The current flows from the active electrode to the neutral electrode. The current density is highest in the immediate vicinity of the active electrode and this is where coagulating or parting of the tissue takes place.

When using neutral electrodes, one must ensure that the contact resistance between the skin and the contacting electrode is not too high. This would lead to severe heating of the biological tissue and occasionally to burns. Recently, the problem has presented itself that more methods have been developed by which relatively large HF currents are applied over longer periods of time. The risk of a burn at the neutral electrode is therefore increased. It should also be noted that, due to the physical conditions, the maximum heating occurs at the edge regions of the neutral electrode. The risk of burning is therefore particularly high in these edge regions.

Large area neutral electrodes are used to prevent unwanted damage to the tissue by contributing to reducing the current density in the immediate vicinity of the neutral electrode. Monitoring devices, which recognize partial detachment of the neutral electrode and react to this event accordingly, also exist.

Currently, applicable standards prescribe tests that limit a temperature rise at the neutral electrode, upon application of a particular current over a predetermined time period, to a maximum value.

With the solutions that are conventionally selected, a further problem exists wherein the area of the neutral electrode cannot be increased without restriction, otherwise the application would no longer be practical. Suitable placement of the neutral electrode becomes more difficult with its increasing size. Furthermore, in pediatric surgery, narrow limits apply to the size of the electrode.

The monitoring devices known and used today can only indirectly determine the degree of risk because it is usually only the electrical contact resistance between the neutral electrode and the patient that is measured, which only has a vague correlation to the application area.

As mentioned above, the current densities at the neutral electrode are not evenly distributed. For example, severe heating leading to burning can occur at the edges. Monitoring these local effects is extremely difficult.

### SUMMARY

Based upon this prior art, it is an object of the embodiments disclosed herein to provide an improved neutral electrode device. In particular, damage to the tissue by the HF current in the region of the neutral electrode is to be prevented. Furthermore, a correspondingly improved electrosurgical instrument and contact medium are disclosed.

In particular, the object is achieved by a neutral electrode device for application of an HF current to a biological tissue, wherein the device comprises at least one latent heat accumulator for absorbing heat.

The core of the embodiments actively counteract burning because a temperature rise in a critical region is sufficiently prevented by cooling effects. The neutral electrode device according to the disclosed embodiments comprises a latent heat accumulator for this purpose, which absorbs the thermal peaks that occur during treatment and accumulates them over a long time period. It is therefore possible to absorb brief temperature rises. The accumulated thermal energy can be released during the operation or following the operation. Thus, a thermal safety reserve that effectively prevents critical temperature rises, even during relatively long activation cycles using large currents, can be built into the neutral electrode arrangement.

The neutral electrode device can comprise at least one electrode, particularly made from aluminum, wherein the latent heat accumulator is arranged flat on the electrode. It is advantageous for the latent heat accumulator to be distributed over the whole electrode surface to absorb heat energy. The heat energy can therefore always be absorbed where it arises. It is also possible for the latent heat accumulator to be distributed to conform to the distribution of heat where it arises. For example, a latent heat accumulator of large capacity could be provided at the edges of the electrode.

On application, the latent heat accumulator can be arranged on a side of the electrode facing away from the biological tissue. The latent heat accumulator therefore does not interfere with the application of the HF current. In particular, it does not act as a resistor, which can lead to an unwanted rise in the temperature of the neutral electrode device. Direct contact between the latent heat accumulator and the biological tissue is also prevented. Possible compatibility problems with the hydrogel used on application can also be prevented. Reliable uptake of the thermal energy can be achieved by direct contacting of the electrode.

The neutral electrode device can comprise at least one supporting fiber layer having phase change materials (PCMs). It is usual to apply the electrodes of the neutral electrode device to a flexible woven fabric or supporting non-woven fabric to enable optimal contact with the individual anatomical structures of the patient. The phase change material can complement or replace this supporting non-woven fabric. For example, PCM fibers can be worked into the supporting non-woven fabric. Alternatively, the supporting non-woven fabric can be replaced with PCM fibers.

Alternatively or additionally, the neutral electrode device can comprise a latent heat accumulator having a cooling cushion. The cooling cushion could be applied, for example, to the neutral electrode device. In this way, large quantities of the material of the latent heat accumulator could be made. A suitable cooling cushion could also be reused. Handling of the cooling cushion is very simple. The cushion can be exchanged during the operation. When overheating of the electrodes would occur during the operation, such that the capacity of the latent heat accumulator is used up, the exchange would be possible without any substantial difficulty.

The object of the disclosed embodiments is also achieved with an electrosurgical instrument for coagulating and/or cutting tissue, wherein the instrument comprises a neutral electrode device as described above. The same advantages result therefrom.

The object of the disclosed embodiments can also be solved with a contact medium for improving the electrical contact between an electrode and a biological tissue, wherein the contact medium comprises at least one latent heat accumulator for absorbing heat, and a conducting substance. It is therefore possible to provide a contact medium for neutral electrodes that improves the contact between the electrode and the biological tissue. The contact medium also comprises a latent heat accumulator, which is suitable for absorbing the arising heat. A particular advantage of the contact medium is that the latent heat accumulator can absorb the thermal energy of the electrode and the electrical tissue. No adaptation of the neutral electrode is necessary.

The conducting substance can come from the viscoelastic group of fluids. In particular, the conducting substance can be a hydrogel. Hydrogel is particularly well suited for improving the electrical contact between electrodes and biological tissue. Hydrogel can also be easily applied. Phase change material can also be mixed into the hydrogel, for example, in powder form. It is also possible to use a two-layered gel, where the lower layer that is in contact with the biological tissue is the conductive substance or the hydrogel and the upper layer is a phase change material in gel form.

The object of the disclosed embodiments is also achieved by the use of a latent heat accumulator for cooling a neutral electrode, particularly for HF surgical applications.

This also has similar advantages to those described above.

The above-described latent heat accumulator can be a phase change material, particularly from the paraffin group of materials.

For ease of processing, the phase change material can be encapsulated in silicate or synthetic fibers.

The latent heat accumulator can have a melting point that is lower than a maximum temperature at which thermal damage to biological tissue would occur. The latent heat accumulator is therefore only activated once the biological tissue or the neutral electrode device approaches a critical temperature. In this way, the resources of the latent heat accumulator can be optimally utilized.

The maximum temperature can be lower than 70° C., particularly lower than 60° C., 50° C., 40° C., 35° C., or 30° C.

Suitably, the melting point of the material used should be chosen so that it is higher than the surface temperature of the biological tissue. In particular, the melting point should be higher than a minimum temperature, particularly higher than 20° C., or 25° C.

### BRIEF DESCRIPTION OF THE DRAWINGS

Embodiments of the invention will now be described in greater detail with reference to the drawings, in which:

FIG. 1 illustrates a monopolar electrosurgical instrument for coagulating and/or cutting tissue;

FIG. 2 illustrates a neutral electrode comprising hydrogel, wherein PCM is added to the hydrogel;

FIG. 3 illustrates a neutral electrode with an additional PCM layer;

FIG. 4 illustrates a neutral electrode with a supporting non-woven fabric made from PCM fibres; and

FIG. 5 illustrates a neutral electrode with a PCM cushion.

### DETAILED DESCRIPTION

In the description that follows, the same reference signs are used for the same and similarly acting parts.

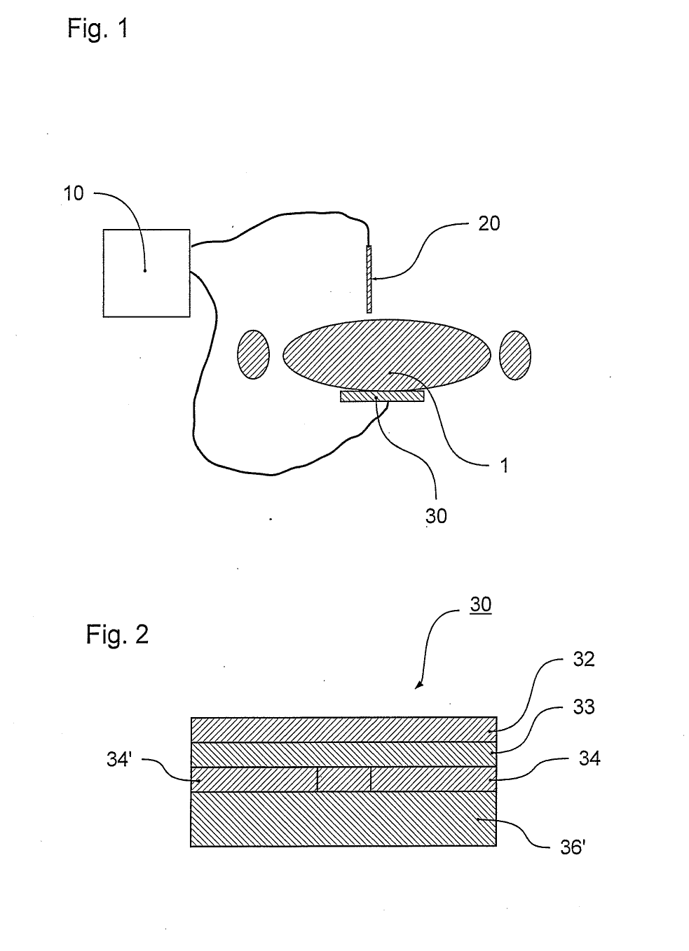

FIG. 1 shows an electrosurgical device comprising an HF generator 10, a monopolar instrument 20 and a neutral electrode arrangement 30. On application of HF current, a voltage is applied between the monopolar instrument 20 and the neutral electrode 30. The HF treatment current flows through the body being treated, a torso 1 in the illustrated embodiment. The current density in the immediate vicinity of the monopolar instrument 20 is high such that the tissue being contacted is coagulated or parted.

To avoid burning at the neutral electrode 30, according to the illustrated embodiment, a latent heat accumulator should be provided. As shown in FIGS. 2-5, neutral electrode arrangement 30 usually comprises three layers. Adjacent to the biological tissue is the electrode layer, comprising a plurality of mutually electrically separated electrodes 34, 34′. A PET support 33, which is glued to a supporting non-woven fabric 32, is provided on the electrodes 34, 34′.

A hydrogel 36 (or 36′) is applied to improve the electrical contact between the biological tissue and the electrodes 34, 34′.

In the first exemplary embodiment (see FIG. 2), the latent heat accumulator is contained in the hydrogel 36. FIG. 2 shows a hydrogel 36′ with PCM components. The PCM is in powder form and is mixed into the hydrogel 36.

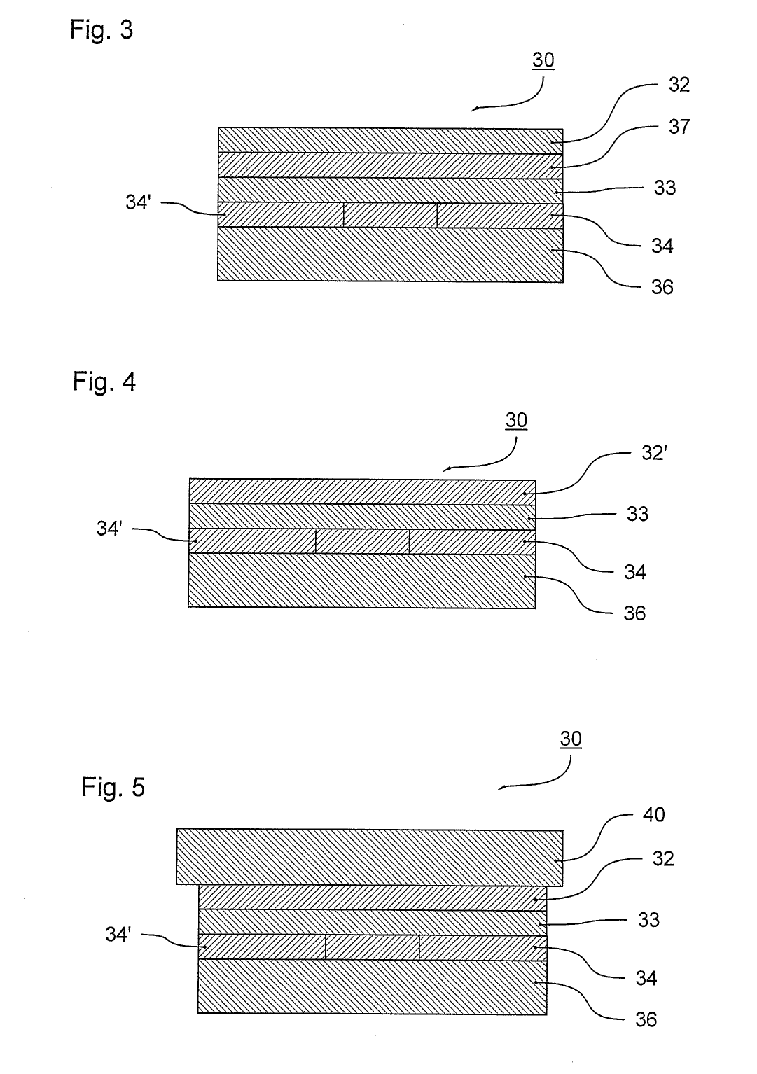

In a second exemplary embodiment, shown in FIG. 3, the neutral electrode arrangement 30 has an additional PCM layer 37 arranged between the supporting non-woven fabric 32 and the PET support 33. It is also possible to use a metal alloy with a low melting point. A thermally conductive contact to the electrodes 34, 34′ can be made via the PET support 33. The heat arising at the electrodes 34, 34′ can therefore be absorbed by the PCM layer 37. Alternatively, the PET support 33 can be dispensed with or replaced with PCM.

In a third exemplary embodiment (see FIG. 4), the neutral electrode arrangement 30 has an adapted supporting non-woven fabric. This is a supporting non-woven fabric 32′ with PCM fibres. These fibres are known in the textile industry and can readily be processed into a woven fabric structure. Direct contacting of the biological tissue with the PCM is prevented by use of the supporting non-woven fabric 32′ with PCM fibres. Low demands can therefore be placed on the tolerability of the PCM used.

In a fourth exemplary embodiment (see FIG. 5), the neutral electrode arrangement 30 is complemented with a PCM cushion 40. The cushion 40 can be applied over a large area on the neutral electrode arrangement 30 after placement of the neutral electrode arrangement 30 on the biological tissue and can serve as, the latent heat accumulator.

The cushion 40 is very easy to use and large amounts of the phase change material can be arranged therein. The PCM cushion 40 therefore has a large storage capacity.

Some concrete exemplary embodiments of the use of PCM in conjunction with the neutral electrode arrangement 30 have been described. However, it is also possible to combine the individual exemplary embodiments with one another. For example, the hydrogel 36′ with PCM components can be used in conjunction with the PCM cushion 40.

1-14. (canceled)

15. A neutral electrode device for applying a high frequency (HF) current to a biological tissue, said device comprising at least one latent heat accumulator for absorbing heat.

16. The neutral electrode device according to claim 15, further comprising at least one electrode, wherein the at least one latent heat accumulator is arranged on the at least one electrode.

17. The neutral electrode device according to claim 16, wherein the at least one electrode is made from aluminum.

18. The neutral electrode device according to claim 16, wherein the at least one latent heat accumulator is arranged flat on the at least one electrode.

19. The neutral electrode device according to claim 16, wherein upon application, the at least one latent heat accumulator is arranged on a side of the at least one electrode facing away from the biological tissue.

20. The neutral electrode device according to claim 15, further comprising at least one supporting fiber layer with phase change material.

21. The neutral electrode device according to claim 15, wherein the at least one latent heat accumulator comprises a cooling cushion.

22. The neutral electrode device according to claim 15, wherein the at least one latent heat accumulator comprises a phase change material.

23. The neutral electrode device according to claim 15, wherein the at least one latent heat accumulator comprises a phase change material a paraffin group of materials.

24. The neutral electrode device according to claim 15, wherein the at least one latent heat accumulator has a melting point that is lower than a maximum temperature at which thermal damage to biological tissue occurs.

25. An electrosurgical instrument for coagulating and/or cutting tissue, said instrument comprising a neutral electrode device comprising a latent heat accumulator for absorbing heat.

26. The electrosurgical instrument according to claim 25, wherein the electrode device further comprises at least one electrode, wherein the latent heat accumulator is arranged on the at least one electrode.

27. The electrosurgical instrument according to claim 25, wherein upon application, the at least one latent heat accumulator is arranged on a side of the at least one electrode facing away from the biological tissue.

28. The electrosurgical instrument according to claim 25, wherein the neutral electrode device further comprises at least one supporting fiber layer with phase change material.

29. The electrosurgical instrument according to claim 25, wherein the latent heat accumulator comprises a cooling cushion.

30. The electrosurgical instrument according to claim 25, wherein the at least one latent heat accumulator comprises a phase change material.

31. The electrosurgical instrument according to claim 25, wherein the at least one latent heat accumulator comprises a phase change material a paraffin group of materials.

32. The electrosurgical instrument according to claim 25, wherein the at least one latent heat accumulator has a melting point that is lower than a maximum temperature at which thermal damage to biological tissue occurs.

33. A contact medium for improving electrical contact between an electrode and a biological tissue, said medium comprising:
at least one latent heat accumulator for absorbing heat; and
a conducting substance.

34. The contact medium according to claim 33, wherein the conducting substance is selected from a viscoelastic group of fluids.

35. The contact medium according to claim 34, wherein the conducting substance is hydrogel.

36. A method of cooling a neutral electrode, particularly for HF surgical applications, said method comprising applying a latent heat accumulator to the neutral electrode.

37. The method of claim 36, wherein the latent heat accumulator comprises a phase change material.

38. The method of claim 37, wherein the latent heat accumulator comprises a phase change material a paraffin group of materials.

39. The method of claim 37, wherein the phase change material is encapsulated in silicate or synthetic fibres.

40. The method of claim 36, wherein the latent heat accumulator has a melting point that is lower than a maximum temperature at which thermal damage to biological tissue occurs.

41. The method of claim 40, wherein the maximum temperature is lower than 70° C.

42. The method of claim 40, wherein the maximum temperature is lower than 60° C.

43. The method of claim 40, wherein the maximum temperature is lower than 50° C.

44. The method of claim 40, wherein the maximum temperature is lower than 40° C.

45. The method of claim 40, wherein the maximum temperature is lower than 35° C.

46. The method of claim 40, wherein the maximum temperature is lower than 30° C.

47. The method of claim 40, wherein the melting point is higher than a minimum temperature of about 20° C.

48. The method of claim 40, wherein the melting point is higher than a minimum temperature of 25° C.
